(19) United States
(12) Patent Application Publication
(10) Pub. No.: US 2024/0238603 A1
(43) Pub. Date: Jul. 18, 2024

(54) Systems and Methods for Multisite Stimulation Using a Double-Tuned Transmitter Coil

(71) Applicant: The Regents of the University of California, Oakland, CA (US)

(72) Inventors: Aydin Babakhani, Los Angeles, CA (US); Iman Habibagahi, Los Angeles, CA (US)

(73) Assignee: The Regents of the University of California, Oakland, CA (US)

(21) Appl. No.: 18/414,155

(22) Filed: Jan. 16, 2024

Related U.S. Application Data
(60) Provisional application No. 63/479,919, filed on Jan. 13, 2023.

(51) Int. Cl. A61N 1/378; A61N 1/372; H02J 50/10; H02J 50/20; H03H 7/38

(52) U.S. Cl. CPC A61N 1/3787; A61N 1/37223; H02J 50/10; H02J 50/20; H03H 7/38

(57) ABSTRACT

Systems and methods for a dual-input double-tuned transmitter (Tx) configured to wirelessly power and control a plurality of implantable stimulators using radio frequency (RF) signals in accordance with embodiments of the invention are illustrated. One embodiment includes a transmitter, where the transmitter includes a matching network configured to isolate two input signals of different frequencies, wherein the matching network includes a lumped element frequency trap comprising at least one inductor and a plurality of capacitors. The transmitter further includes a Tx coil, wherein the Tx coil is configured to transmit two RF signals at the frequencies of the input signals with a difference in frequency by a factor of 10% or more.

### CROSS-REFERENCE TO RELATED APPLICATIONS

The current application claims the benefit of and priority under 35 U.S.C. § 119(e) to U.S. Provisional Patent Application No. 63/479,919 entitled “Systems and Methods for Multisite Stimulation Using a Double-Tuned Transmitter Coil” filed Jan. 13, 2023. The disclosure of U.S. Provisional Patent Application No. 63/479,919 is hereby incorporated by reference in its entirety for all purposes.

### FIELD OF THE INVENTION

The present invention generally relates to multisite stimulation systems that can use a double-tuned transmitter coil for multisite stimulation.

### BACKGROUND

Traditional battery-powered implantable devices face limitations such as large size and compromised safety. Wireless power transfer (WPT) can be a popular solution to remove the implanted battery by adding an external wearable transmitter. Multisite powering and controlling a network of implants can have different applications in biventricular heart pacing, bilateral vagus nerve stimulation, and spinal cord stimulation, among other biomedical applications.

### SUMMARY OF THE INVENTION

The description and claims will be more fully understood with reference to the following figures and data graphs, which are presented as exemplary embodiments of the invention and should not be construed as a complete recitation of the scope of the invention.

Systems and methods for a dual-input double-tuned transmitter (Tx) configured to wirelessly power and control a plurality of implantable stimulators using radio frequency (RF) signals in accordance with embodiments of the invention are illustrated. One embodiment includes a transmitter, where the transmitter includes a matching network configured to isolate two input signals of different frequencies, wherein the matching network includes a lumped element frequency trap comprising at least one inductor and a plurality of capacitors. The transmitter further includes a Tx coil, wherein the Tx coil is configured to transmit two RF signals at the frequencies of the input signals with a difference in frequency by a factor of 10% or more.

In a further embodiment, the matching network comprises a plurality of capacitors comprising a first capacitor C₁, a second capacitor C₂, a third capacitor C₃, a fourth capacitor C₄, a fifth capacitor C₅, and a trap inductor L₂.

In still another embodiment, the third capacitor C₃ is connected in series to one of the two input signals and the fourth capacitor C₄ is connected in series to the second of the two input signals, wherein the first capacitor C₁ and second capacitor C₂ are connected in series between the third capacitor C₃ and the fourth capacitor C₄, and in parallel with a Tx coil, wherein the fifth capacitor C₅ is connected between one of the two input signals and the third capacitor, wherein the matching network comprises a lumped element frequency trap comprising the trap inductor L₂ connected in parallel with the second capacitor C₂, configured to isolate the two inputs.

In a still further embodiment, the trap frequency is chosen between the frequencies of the two inputs.

In yet another embodiment, an inductance ratio between the coil and the trap inductor is close to 1.

In a yet further embodiment, the lumped element frequency trap is configured using the second capacitor C₂ and the trap inductor L₂.

In another additional embodiment, the inductance of the Tx coil is between 1 μH to 1.25 μH.

In a further additional embodiment, the Tx coil operates at 13.56 and 40.68 MHz industrial, scientific, and medical (ISM bands).

In another embodiment again, the plurality of capacitors provides isolation of approximately 17.7 dB and matching of approximately −26.2 at 13.56 MHz and approximately −21.5 dB at 40.68 MHz.

In a further embodiment again, further including two bidirectional ports capable of simultaneously transmitting and receiving signals.

In still yet another embodiment, the trap frequency is tuned by adjusting either C₂ or C₁.

In still another additional embodiment, capacitors C₃ and C₄ are high-Q configured to match resonated inductors to 50Ω.

In a still further additional embodiment, capacitor C₅ is used to improve high-frequency isolation between the two transmitted RF signals.

One embodiment includes a wirelessly powered and controlled implantable stimulator configured to receive RF signals from a transmitter and provide stimulation using received RF signals. The stimulator includes a tuning capacitor Cₜ configured to tune the stimulator to receive RF signals of a certain frequency, a receiving (Rx) coil configured to receive RF signals of the certain frequency, and a chip configured to convert the received RF signals to a power signal and a control signal. The stimulator further includes a storage capacitor Cₛₜᵣ configured to store rectified voltage based on the power signal, and at least one electrode configured to stimulate a target area based on the control signal and stored voltage.

In still another embodiment again, the stimulator further includes a charge balancer comprising a discharge resistor R_{dis} and a blocking capacitor C_{blk}.

In a yet further additional embodiment, the power signal is a function of the geometries of the Rx coil.

In yet another embodiment again, the implantable stimulator can be tuned to operate at 13.56 MHz or 40.68 MHz.

In still yet another embodiment again, the chip generates feedback based on provided stimulation and provide the generated feedback to the transmitter.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A illustrates a prior art two-coil system.

FIG. 1B illustrates a system that uses a single transmit (Tx) coil with dual inputs at 13.56 MHz and 40.68 MHz in accordance with an embodiment of the invention.

FIG. 2 illustrates a fabricated circular transmitter (Tx) coil with a diameter of 62 mm connected to a matching network circuit in accordance with an embodiment of the invention.

FIG. 3 illustrates a circuit schematic of a matching network in accordance with an embodiment of the invention.

FIGS. 4A-D illustrate simulated and measured matching and isolation results of a double-tuned transmitter coil in accordance with an embodiment of the invention.

FIG. 5 illustrates a coil quality factor, self-resonance frequency (SRF), and matching network parameters in accordance with an embodiment of the invention.

FIG. 6 illustrates a schematic of a printed circuit board (PCB) implantable stimulator in accordance with an embodiment of the invention.

FIG. 7 illustrates details of the implantable stimulator and component values in accordance with an embodiment of the invention.

FIG. 8 illustrates a process for providing multisite stimulation using wirelessly powered implantable stimulators in accordance with an embodiment of the invention.

FIG. 9 illustrates a schematic of a verification setup in accordance with an embodiment of the invention.

### DETAILED DESCRIPTION OF THE DRAWINGS

Implantable devices are small electronic devices that may be surgically implanted into the body to provide therapeutic benefits for a variety of medical conditions. They can provide significant relief for patients who suffer from chronic pain or other medical conditions that are difficult to manage with traditional therapies. In many situations, implantable devices can help reduce the patient's need for invasive surgeries or frequent visits to the doctor's office. Additionally, implantable devices can improve the quality of life for patients by providing more effective pain relief, reducing symptoms, and improving overall health and well-being.

Implantable devices are typically powered by batteries and can be programmed to deliver specific amounts of medication or electrical stimulation to targeted areas of the body. However, current battery-powered implantable devices may be limited by these batteries in various ways. The batteries may need to be replaced periodically through invasive surgery, which can be risky and inconvenient for the patient. The need for invasive surgeries to replace batteries also essentially defeats the purpose of using an implantable device. Additionally, the batteries can add significant weight and bulk to the implantable device, which can make it more difficult to implant and can cause discomfort for the patient. Finally, the batteries can potentially leak or rupture, which can cause harm to the patient.

Wireless power transfer (WPT) is a technology that allows power to be transmitted wirelessly as a signal from a transmitter to a receiver without the need for physical contact or electrical conductors. In the context of implantable devices, WPT can be used to power and control the implantable devices remotely, which greatly reduces the need for a battery that would require periodic maintenance. By removing the battery, current wirelessly-powered implantable devices can become smaller, lighter, and more comfortable for patients to use. Additionally, the risk of battery leakage or rupture may be reduced, which can improve the overall safety of the implantable device. Furthermore, without the need for battery replacement, the lifespan of the device can be extended, and the device can be programmed to deliver more precise and targeted stimulation to specific areas of the body, improving its overall effectiveness.

Current wirelessly-powered implantable devices are able to utilize WPT to provide many benefits to patients. However, they are typically only able to operate under a single frequency. This generally precludes current wirelessly-powered implantable devices from being used to achieve multisite control and stimulation. Multisite stimulation using wirelessly powered implantable devices is a technique used in biomedical applications where multiple sites of the body are stimulated simultaneously. This technique has remarkable applications that can improve patients' quality of life, including biventricular heart pacing, bilateral vagus nerve stimulation, and spinal cord stimulation. However, multisite stimulation and control are nearly impossible when the implanted devices each operate under a single different frequency. It is possible for cross-talk to occur between multiple implantable devices, where the receivers of the devices pick up each other's signals. This can result in insufficient powering and/or inaccurate control of the implantable devices, both of which can result in significant consequences for the patients.

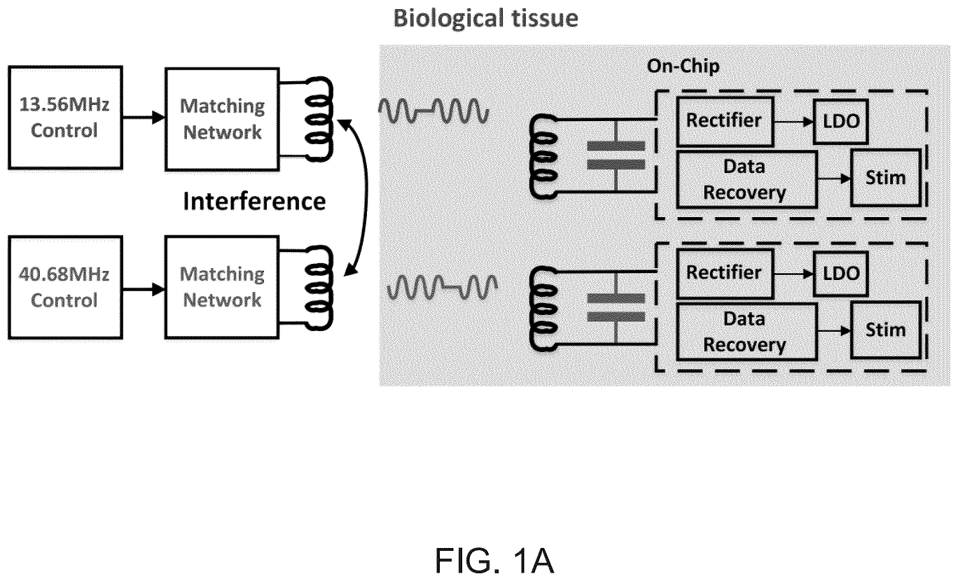

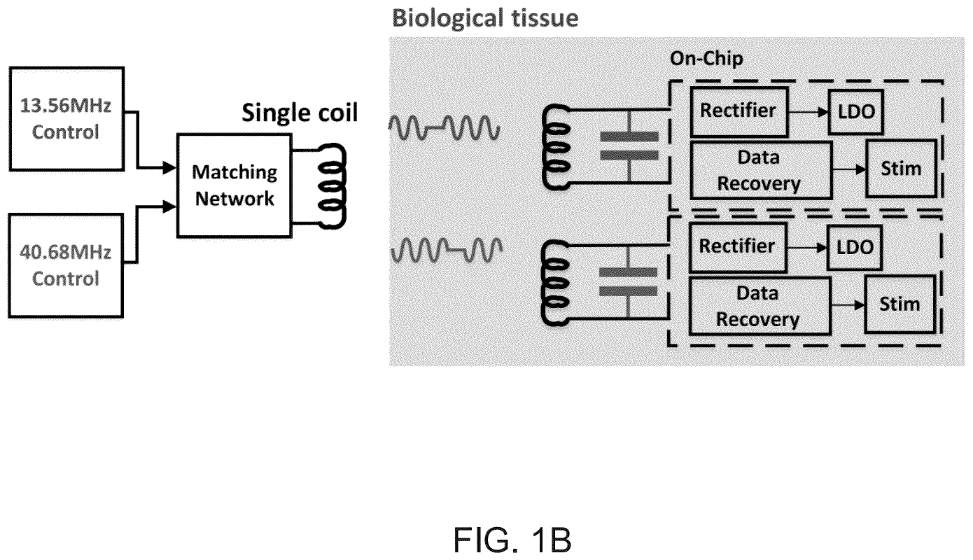

Attempts at transmission of multi-band signals have been hindered by the difficulty of minimizing the interference between the multiple transmitted signals. In certain solutions, transmitters with multiple transmission coils are used to transmit multi-band signals, where each coil can be configured to transmit power and control signals at a certain frequency. FIG. 1A illustrates a prior art two-coil system for dual-band transmission where each coil is matched to transmit at a different frequency. However, the use of multiple transmission coils can cause the transmitter to be bulky and not easily deployable. Other attempts have included a pattern detection scheme for multisite stimulation using a physical unclonable function (PUF) with dual transmission coils. This scheme can require more power to recover the clock and data on the implant side. Another prior passcode detection scheme using chip pins has been described which similarly requires more chip area and power. In certain prior techniques, multiple-channel chips and electrode multiplexing were proposed. However, these techniques have led to larger electrode sizes and larger implantable devices, which can make these devices difficult to use in applications such as heart pacing and vagus nerve stimulation. A single coil, single input dual-band transmission system using a lumped element frequency trap has also been attempted, but such a system includes large multi-band drivers and coils, which cannot be easily realized for implantable or wearable devices.

Systems and methods in accordance with many embodiments of the invention can address these challenges by providing a novel multisite stimulation system using a double-tuned transmitter (Tx) that can improve the efficiency and reliability of multisite stimulation using WPT. In many embodiments, stimulation systems include a single Tx coil that is double-tuned using two input signals of different frequencies such that the Tx coil is capable of transmitting power and control signals at both input frequencies(e.g., at 13.56 MHz and 40.68 MHz industrial, scientific, and medical (ISM) bands) for multisite biomedical applications. Systems and methods in accordance with several embodiments can remove the need for two separate Tx coils, which can reduce system size and unwanted couplings.

A system that uses a single Tx coil with dual inputs at 13.56 MHz and 40.68 MHz in accordance with an embodiment of the invention is illustrated in FIG. 1B. In numerous embodiments, Tx coils are connected to a novel matching network circuit that is capable of maintaining dual-band transmission while preserving good isolation between the two signals that are being transmitted. Previously, multiband drivers have been used to maintain isolation between the multiple bands of signals during transmission. However, multiband drivers are typically expensive. Systems and methods in accordance with many embodiments can provide an inexpensive solution to maintain dual-band transmission with good isolation without using a multiband driver. In several embodiments, matching networks may include a lumped element frequency trap to allow the single Tx coil to resonate at two different frequencies. Matching networks can include bidirectional ports, which can convert the Tx into a transceiver capable of sending signals and receiving feedback from implantable devices at the same time. In many embodiments, Tx coils can achieve measured matchings of −26.2 dB and −21.5 dB and isolation of −17.7 dB and −11.7 dB at 13.56 MHz and 40.68 MHz, respectively. In many embodiments, Tx coils can power up and control two implantable stimulators at a distance of 2 cm with 800 mW of power at each port.

In some embodiments, a 3 mm×15 mm flexible coil can be used as an implantable receiver (Rx) coil. Implantable stimulators in accordance with several embodiments can have a width of approximately 3 mm, which makes them favorable for different biomedical applications, including heart-pacing applications, among others.

### Tx Coil Designs

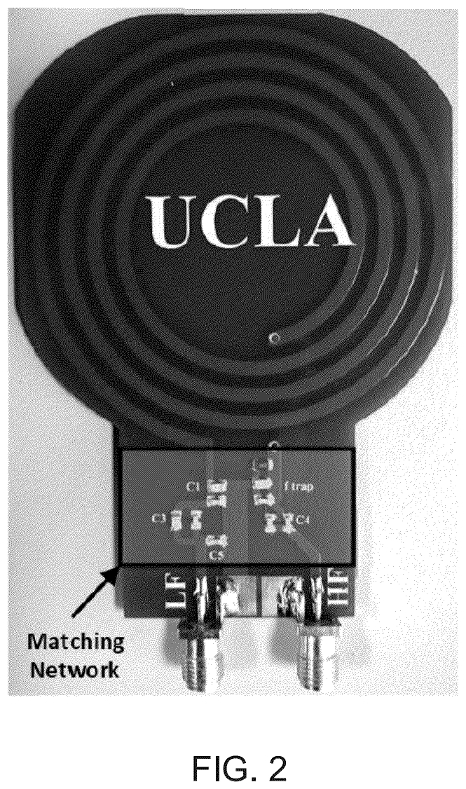

In many embodiments, double-tuned Tx coils capable of transmitting two signals of different input frequencies can be fabricated on a 1.6 mm FR4 substrate. FIG. 2 illustrates a fabricated circular Tx coil with a diameter of 62 mm connected to a matching network circuit in accordance with an embodiment of the invention.

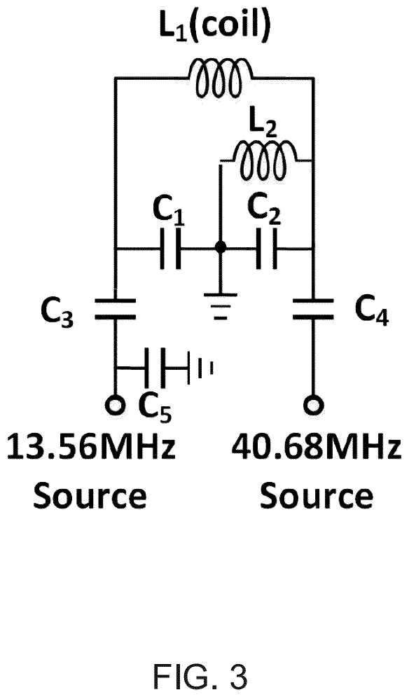

FIG. 3 illustrates a circuit schematic of a matching network in accordance with an embodiment of the invention. In many embodiments, matching networks include at least five capacitors C₁, C₂, C₃, C₄, and C₅. Matching network circuits may further include a Tx coil L₁ and a trap inductor L₂. Tx coil L₁ may be connected in series to capacitor C₃ and then to a frequency source. Tx coil L₁ may be connected in series to capacitor C₄ and then to a different frequency source. Capacitors C₁ and C₂ can be connected in parallel to L₁ between capacitors C₃ and C₄. In many embodiments, a ground node is shared between C₁ and C₂. Trap inductor L₂ may be connected in parallel to capacitor C₂ to form a lumped element frequency trap. Frequency traps can be used to isolate the resonant frequencies of the coil, and thus can allow the Tx coil to function effectively at two different frequencies. In several embodiments, capacitor C₅ may be connected to the frequency source opposite of the frequency trap.

As shown in Equation 1 below, the trap frequency may be chosen to be between the frequencies of the two input signals:

$$13.56 MHz < {f}_{L {C}_{1 , 2}} = \frac{1}{2 π \sqrt{{L}_{1 , 2} {C}_{1 , 2}}} < 40.68 MHz . \qquad ( 1 )$$

In several embodiments, the inductance ratio r_{L}=L₁/L₂ between the coil and the trap is chosen to be approximately 1 to maintain the same efficiency at two frequencies. In many embodiments, the coil can be designed to have an inductance close to 1 μH. The coil inductance can vary from 1 μH to 1.25 μH. In numerous embodiments, the lumped inductance L₂ is 1 μH, and its self-resonance frequency (SRF) is 330 MHz. In some embodiments, C₂ can be chosen to be 28 pF to set the trap frequency f_{LC2} at 30.08 MHz. The same principle can apply to the series LC branch that can include the coil L₁ and C₁. In selected embodiments, C₁ can be tuned to 21.8 pF, which sets trap frequency f_{LC1} at 34.1 MHz. High-Q capacitors C₃ and C₄ can be added to match the resonated inductors to 50Ω. In many embodiments, systems and methods are able to maintain good high-frequency isolation between the different ports of the transmitter by the inclusion of the capacitor C₅.

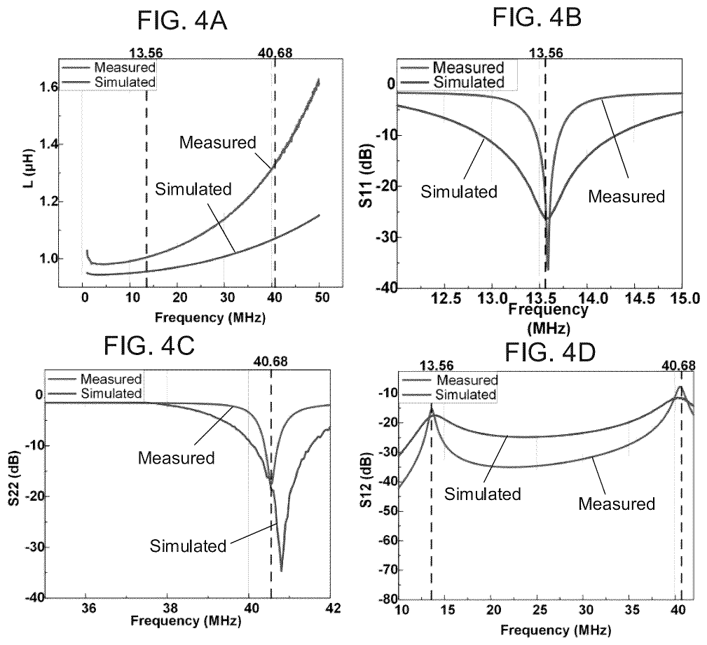

FIGS. 4A-D illustrate simulated and measured matching and isolation results of a dual-band Tx coil with a matching network in accordance with an embodiment of the invention. Specifically, FIG. 4A illustrates the measured and simulated inductance of the Tx coil. FIG. 4B illustrates the measured and simulated low-frequency port matching of the Tx coil. FIG. 4C illustrates the measured and simulated high-frequency port matching of the Tx coil. FIG. 4D illustrates the measured and simulated isolation between ports of the Tx coil. FIG. 4 illustrates the characterization of a double-tuned Tx coil. S11 and S22 are matching S-parameters. S-parameters can indicate how much of the source power is reflected back to the source and therefore not utilized in the transmission and powering of stimulators. S21 can represent the isolation between ports, which indicates how much of low frequency port's power may leak to the high frequency port and vice versa. Based on the S-parameters, FIGS. 4B and 4C demonstrate that low-frequency and high-frequency matching can be better than −26.2 dB and −21.5 dB, and isolation between the ports is −17.7 dB and −11.7 dB, respectively.

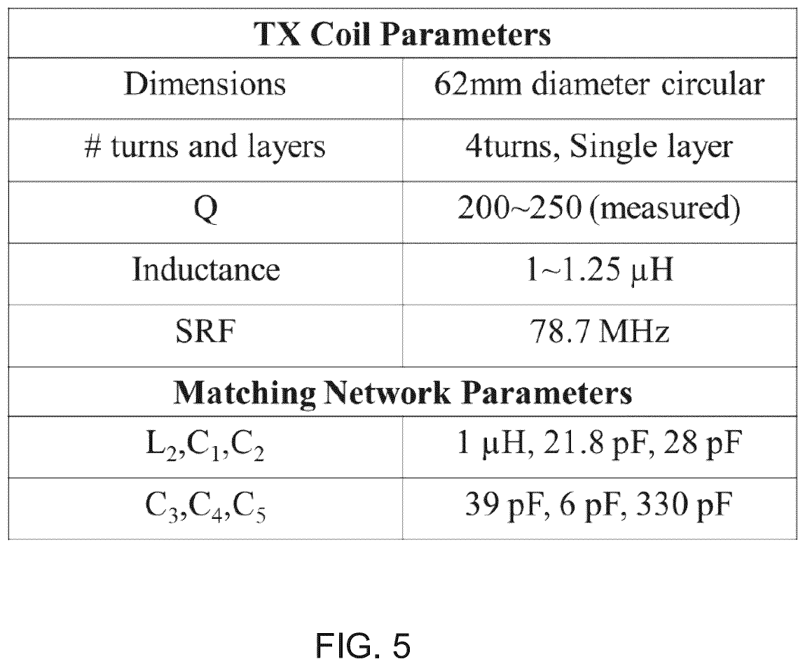

FIG. 5 illustrates a table showing the coil quality factor, SRF, and matching network parameters in accordance with an embodiment of the invention. Parameters illustrated in FIG. 5 may be used to configure similar multisite stimulation systems. In some embodiments, a double-tuned dual input Tx coil configured using the parameters set forth in FIG. 5 can be matched to the frequencies of 40.68 MHz and 13.56 MHz at the high-frequency (HF) and low-frequency (LF) inputs, respectively. Tx coils in accordance with many embodiments can act as both the Tx and Rx coils and can reduce inter-coil couplings compared to a two-coil system with separate Tx and Rx coils.

Although specific examples of transmitters capable of dual-band transmission using a single Tx coil are described above with respect to the figures, any of a variety of transmitters can be utilized to transmit signals of different frequencies using a single Tx coil similar to those described herein as appropriate to the requirements of specific applications in accordance with embodiments of the invention.

### Implantable Stimulator Designs

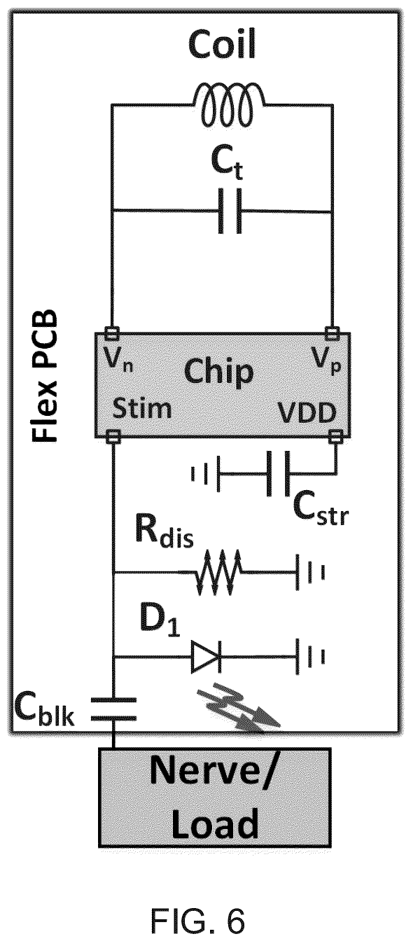

Keeping the size of implantable devices small is generally a desirable outcome when designing implantable devices. Smaller devices can be easier to implant and are more comfortable for patients to use. Smaller devices can be more discreet and less noticeable to others, which can improve the quality of life and self-esteem of patients who require them. Stimulator systems in accordance with many embodiments of the invention include implantable stimulators that can be fabricated on a multi-turn 4-layer 0.15 mm flexible polyimide printed circuit board (PCB). FIG. 6 illustrates a schematic of a PCB implantable stimulator in accordance with an embodiment of the invention. In numerous embodiments, implantable stimulators include at least an Rx coil, a tuning capacitor Cₜ, and a chip capable of controlling stimulation. Implantable stimulators may further include a storage capacitor Cₛₜᵣ, discharge resistor R_{dis}, blocking capacitor C_{blk}, and a light emitting diode (LED) D₁.

Implantable stimulators may be placed in direct contact with nerves requiring stimulation. In several embodiments, Rx coils on the PCB can be tuned to receive a signal at a particular frequency. The Rx coil can be used together with the chip to harvest power by converting the signals of a particular frequency received from the Tx coil and storing it on a storage capacitor. The number of implantable stimulators capable of providing multisite stimulation may depend on the number of bands of signal being transmitted from the Tx coil. The geometry and size of the implantable coils can be determined based on leadless pacing constraints. The notches in the wireless link can be detected by the chip and can define the duration of constant voltage stimulation. Chip designs and internal circuitry details are described in I. Habibagahi, M. Omidbeigi, J. Hadaya, H. Lyu, J. Jang, J. L. Ardell, A. A. Bari, and A. Babakhani, “Vagus nerve stimulation using a miniaturized wirelessly powered stimulator in pigs,” Scientific reports, vol. 12, no. 1, pp. 1−12, 2022 and H. Lyu, M. John, D. Burkland, B. Greet, A. Post, A. Babakhani, and M. Razavi, “Synchronized biventricular heart pacing in a closed-chest porcine model based on wirelessly powered leadless pacemakers,” Scientific reports, vol. 10, no. 1, pp. 1−9, 2020, the relevant disclosures of which are herein incorporated by reference in their entirety.

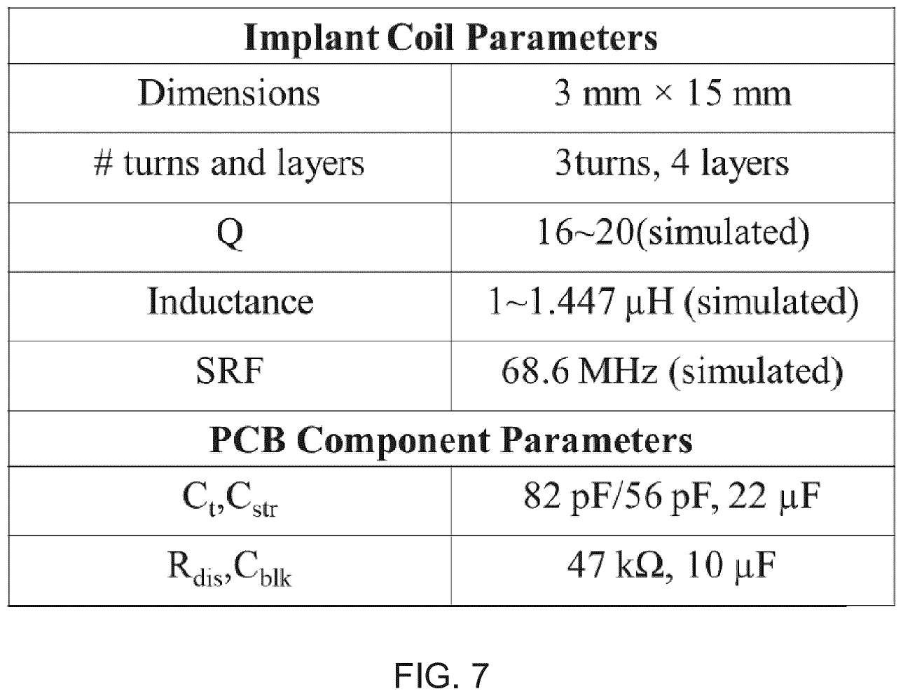

In many embodiments, implantable stimulators can have a width of approximately 3 mm and a length of approximately 41 mm. In many embodiments, an implant can easily fit in 24 French catheters, which illustrates its high potential for deployment in leadless heart pacing. The harvested power may be a function of coil geometries and can decrease with the inverse cube of the distance between the Tx and Rx coils. Depending on the value of the tuning capacitor Cₜ, implantable stimulators can operate at 13.56 MHz when Cₜ is set as 82 pF, or 40.68 MHz when Cₜ is set as 56 pF, for power harvesting. The rectified voltage can be accumulated on the storage capacitor Cₛₜᵣ. In many embodiments, the charge balancing can be done using a blocking capacitor C_{blk} and discharge resistor R_{dis}, and a green LED can also be used to indicate the pulses. FIG. 7 illustrates the details of the implantable stimulator and component values in accordance with an embodiment of the invention.

Although a specific example of an implantable stimulator is illustrated in this figure, any of a variety of implantable stimulators can be utilized to harvest power from transmitted signals and provide stimulation similar to those described herein as appropriate to the requirements of specific applications in accordance with embodiments of the invention.

### Processes for Multisite Stimulation

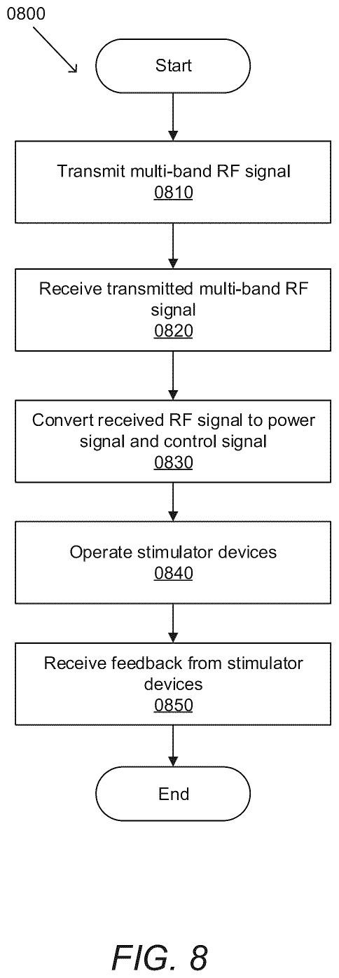

A process for providing multisite stimulation using wirelessly powered implantable stimulators in accordance with an embodiment of the invention is illustrated in FIG. 8. Process 800 transmits (810) a multi-band RF signal. In many embodiments, the multi-band RF signal includes at least two RF signals at different frequencies. The multi-band RF signals may include signals at 13.56 MHz and 40.68 MHz. In several embodiments, the multi-band RF signal is transmitted from a Tx coil on a transmitter to at least one implanted stimulator. A Tx coil may utilize a structure such as those described further above including with respect to FIGS. 1B and 3. In certain embodiments, the transmitter may be implemented as a wearable device such as a necklace that can transmit signals to perform multisite stimulation on the wearer of the device.

Process 800 receives (820) the transmitted multi-band RF signal. The Rx coil on each of the implanted stimulators may pick up the transmitted signal. Each implanted stimulator may be tuned to a particular frequency such that the Rx coils on each of the implanted stimulators can pick up transmitted signals of the particular frequency. This way, transmitters are able to power and control multiple implanted stimulators by transmitting signals of more than one frequency. An implanted stimulator may utilize a structure such as those described further above including with respect to FIG. 6.

Process 800 converts (830) the received multi-band RF signal to a power signal and a control signal. In many embodiments, the implanted stimulators include chips containing power-harvesting circuits that are configured to convert received RF signals into power signals to power the implanted stimulator. The chips on the implanted stimulators can obtain a control signal based on the received RF signal. The control signal can be used to control the manner in which stimulation is to be administered.

Process 800 operates (840) the implanted stimulators. In numerous embodiments, the implanted stimulators operate by providing stimulation to the sites where the stimulators are implanted based on the obtained control signal.

Process 800 receives (850) feedback from the implanted stimulators. Implanted stimulators in accordance with several embodiments can provide feedback based on the stimulation that was provided to the transmitter through the Tx coils. Feedback may be encoded as RF signals and transmitted to the transmitter. Transmitters in accordance with some embodiments of the invention can adjust the power and control signal to be transmitted for the next stimulation based on the feedback.

While specific processes for multisite stimulation using wirelessly powered implantable stimulators are described above, any of a variety of processes can be utilized to perform multisite stimulation as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### System Verification

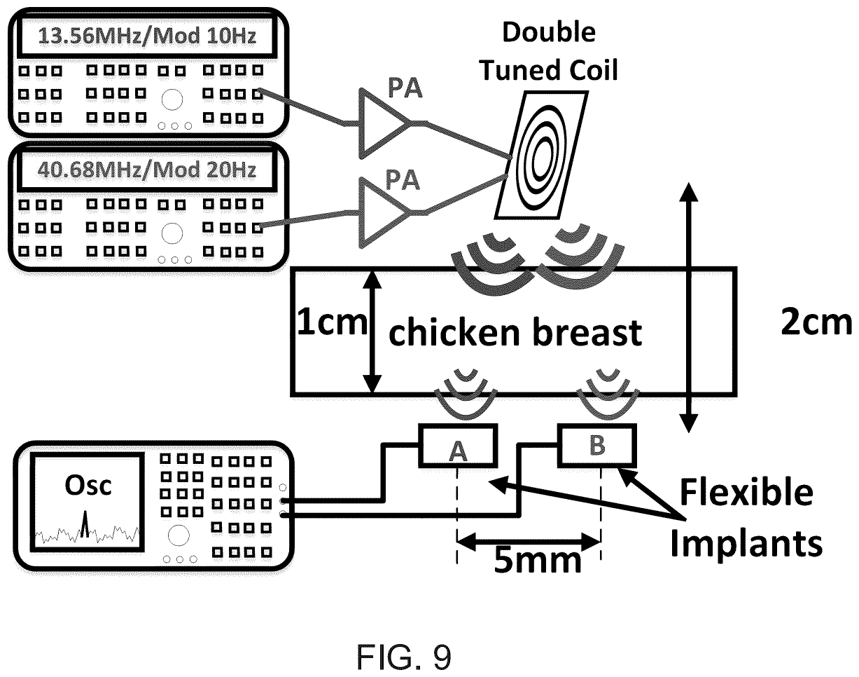

To test dual input wireless operation, two implants tuned at 13.56 MHz and 40.68 MHz, were put under 1 cm of chicken breast with a spacing of 5 mm. Two ports of the Tx coil were excited with 800 mW of power simultaneously. The pulse modulation on each port has a notch duration of 500 μs and a repetition rate of 20 Hz and 10 Hz on 40.68 MHz and 13.56 MHz ports, respectively. The internal pulse modulation option on Agilent E4428C sources was used for modulation. Two power amplifiers for each port were used to reach an output power of 800 mW. FIG. 9 illustrates a schematic of the verification setup in accordance with an embodiment of the invention. FIG. 9 demonstrates the setup where two implantable stimulators are wirelessly powered and controlled at a distance of 2 cm in accordance with an embodiment of the invention. This measurement illustrates WPT and control for two implantable devices using a single Tx coil at two frequencies in accordance with an embodiment of the invention. Systems and methods in accordance with many embodiments can include only one Tx coil and can be used for miniaturized low-power implantable devices suitable for biventricular pacing. Many embodiments can include a single double-tuned Tx antenna and miniaturized implantable stimulators operating at 13.56 MHz and 40.68 MHz ISM bands.

Although specific methods of multisite stimulation using a single double-tuned Tx coil are discussed above, many different stimulation methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A dual-input double-tuned transmitter (Tx) configured to wirelessly power and control a plurality of implantable stimulators using radio frequency (RF) signals, the transmitter comprising:
a matching network configured to isolate two input signals of different frequencies, wherein the matching network comprises a lumped element frequency trap comprising at least one inductor and a plurality of capacitors; and
a Tx coil, wherein the Tx coil is configured to transmit two RF signals at the frequencies of the input signals with a difference in frequency by a factor of 10% or more.

2. The transmitter of claim 1, wherein the matching network comprises a plurality of capacitors comprising a first capacitor C₁, a second capacitor C₂, a third capacitor C₃, a fourth capacitor C₄, a fifth capacitor C₅, and a trap inductor L₂.

3. The transmitter of claim 2, wherein the third capacitor C₃ is connected in series to one of the two input signals and the fourth capacitor C₄ is connected in series to the second of the two input signals, wherein the first capacitor C₁ and second capacitor C₂ are connected in series between the third capacitor C₃ and the fourth capacitor C₄, and in parallel with a Tx coil, wherein the fifth capacitor C₅ is connected between one of the two input signals and the third capacitor, wherein the matching network comprises a lumped element frequency trap comprising the trap inductor L₂ connected in parallel with the second capacitor C₂, configured to isolate the two inputs.

4. The transmitter of claim 1, wherein the trap frequency is chosen between the frequencies of the two inputs.

5. The transmitter of claim 1, wherein an inductance ratio between the coil and the trap inductor is close to 1.

6. The transmitter of claim 1, wherein the lumped element frequency trap is configured using the second capacitor C₂ and the trap inductor L₂.

7. The transmitter of claim 1, wherein the inductance of the Tx coil is between 1 μH to 1.25 μH.

8. The transmitter of claim 1, wherein the Tx coil operates at 13.56 and 40.68 MHz industrial, scientific, and medical (ISM bands).

9. The transmitter of claim 1, wherein the plurality of capacitors provides isolation of approximately 17.7 dB and matching of approximately −26.2 at 13.56 MHz and approximately −21.5 dB at 40.68 MHz.

10. The transmitter of claim 1, further comprising two bidirectional ports capable of simultaneously transmitting and receiving signals.

11. The transmitter of claim 1, wherein the trap frequency is tuned by adjusting either C₂ or C₁.

12. The transmitter of claim 1, wherein capacitors C₃ and C₄ are high-Q configured to match resonated inductors to 50Ω.

13. The transmitter of claim 1, wherein capacitor C₅ is used to improve high-frequency isolation between the two transmitted RF signals.

14. A wirelessly powered and controlled implantable stimulator configured to receive RF signals from a transmitter and provide stimulation using received RF signals, the stimulator comprising:
a tuning capacitor Cₜ configured to tune the stimulator to receive RF signals of a certain frequency;
a receiving (Rx) coil configured to receive RF signals of the certain frequency;
a chip configured to convert the received RF signals to a power signal and a control signal;
a storage capacitor Cₛₜᵣ configured to store rectified voltage based on the power signal; and
at least one electrode configured to stimulate a target area based on the control signal and stored voltage.

15. The implantable stimulator of claim 14, further comprising a charge balancer comprising a discharge resistor R_{dis} and a blocking capacitor C_{blk}.

16. The implantable stimulator of claim 14, wherein the power signal is a function of the geometries of the Rx coil.

17. The implantable stimulator of claim 14, wherein the implantable stimulator is tuned to operate at 13.56 MHz or 40.68 MHz.

18. The implantable stimulator of claim 14, wherein the chip generates feedback based on provided stimulation and provide the generated feedback to the transmitter.
